(19) United States
(12) Patent Application Publication
(10) Pub. No.: US 2024/0425808 A1
(43) Pub. Date: Dec. 26, 2024

(54) A METHOD FOR RAPID GENERATION OF 3D ORGANOTYPIC CELL CLUSTERS

(71) Applicant: THE TRUSTEES OF INDIANA UNIVERSITY, Bloomington, IN (US)

(72) Inventors: Feng GUO, Bloomington, IN (US); Hongwei CAI, Bloomington, IN (US); Zheng AO, Bloomington, IN (US)

(21) Appl. No.: 18/835,051

(22) PCT Filed: Feb. 7, 2023
(86) PCT No.: PCT/US2023/012494

§ 371 Date: Aug. 1, 2024

Related U.S. Application Data
(60) Provisional application No. 63/307,669, filed on Feb. 8, 2022.

(51) Int. Cl. C12N 5/00; C12N 5/0783; C12N 5/09

(52) U.S. Cl. CPC C12N 5/0062; C12N 5/0636; C12N 5/0693; C12N 2503/02; C12N 2513/00; C12N 2521/10

(57) ABSTRACT

Disclosed herein is a system comprising acoustically assembled patient-derived cell clusters that can preserve original tissue compositions, including tumor and immune cell compositions for solid tumors collected from a patient, and model their interactions in 3D microenvironments. Such a system allows for ex vivo manipulations of the system to investigate and predict how the native tissues will responses to treatments, including those of a primary and metastatic patient tumor, in a rapid, scalable, and user-friendly manner.

### CROSS REFERENCE TO RELATED APPLICATION

This application claims priority to U.S. Provisional Patent Application No. 63/307,669, filed on Feb. 8, 2022, the disclosure of which is expressly incorporated herein.

### GOVERNMENT LICENSE RIGHTS

This invention was made with government support under EB030331 awarded by National Institutes of Health. The Government has certain rights in the invention.

### BACKGROUND

Avoiding immune destruction is one of the emerging hallmarks of cancer. Immune suppressive cells such as tumor associated macrophages and myeloid derived suppressor cells (MDSCs) orchestrates local metabolism and cytokine environment to suppress anti-tumor immunity and promote tumor growth. In turn, tumor cells actively reprogram the infiltrated immune cells and induce tumor microenvironment (TME) specific immune suppressive phenotypes, which are distinct from their circulating counterparts. For example, when residing within the TME, myeloid derived suppressor cells (MDSCs) show distinct functions such as suppression of T cells mediated cytotoxicity via production of nitric oxide (NO) and arginase-1 (ARG-1). Clinically, higher numbers of MDSCs are generally associated with resistance to immune checkpoint inhibitor (ICI) treatments. Additionally, animal model studies have also shown that targeting MDSCs can overcome tumors' resistance to ICI.

Currently, it remains a challenge to study human tumor induced MDSC functions and drug responses ex vivo. Once taken out of the context of the tumor microenvironment (TME), key phenotypes of tumor induced MDSCs such as NO and ARG-1 production can be quickly dampened or lost. Genetically engineered humanized mice model may recapitulate human MDSC functions in vivo, yet they fail to reflect patient specific genetics and tumor heterogeneity. Ex vivo cultures of tumor derived cells/organoids with immune cells co-incubation or direct organotypic cultures of tumor segments can preserve TME components partially, yet they generally take ˜1-4 weeks to establish, which may lead to potential loss of key phenotypes of the MDSCs. Additionally, organotypic cultures could lead to stochastic distribution of immune cells inside each tumor section/segments, thus hampering comparable evaluation of multiple drug treatments in parallel. Thus, there is a great need for patient derived tumor models to preserve sensitive TME immune cell phenotypes such as MDSCs whereas allowing for massively parallel profiling of multiple drug treatments with nodes of comparable sizes, cell numbers and cell compositions.

As disclosed herein is a novel assembled patient-derived cell cluster (APCC) that can preserve function and viability of MDSCs. The APCC may be used to evaluate the function and drug responses of patient derived MDSCs.

### SUMMARY

In accordance with one embodiment dissociated cells of an original tissue cab be reassembled acoustically, or through any other applied force to reassemble dissociated cells into clusters of cells. In one embodiment, by applying bio-compatible bulk acoustic waves, patient tumor cell suspensions can be quickly assembly into cell clusters (comprising approximately 100 cells) with uniformed cell compositions and numbers.

As disclosed herein the presently disclosed methods of assembling cell clusters can preserve the viability and functional phenotypes of the MDSCs, including their expression of key immune suppressive genes, inhibition of T cell mediated cytotoxicity, as well as inhibition of pro-inflammatory cytokine secretion. Additionally, the combinational treatment efficacy of MDSC-targeting multi-kinase inhibitor Cabozantinib and anti-PD1 drug Pembrolizumab was investigated in this model system. In one embodiment, by suppressing the MDSCs via Cabozantinib treatment, anti-tumor effect of anti-PD1 on renal cell carcinoma patients' primary tumors can be enhanced by 123±67%. This demonstrated that the APCC disclosed herein could be widely applied to study tumor immunity ex vivo in a fast, scalable, and reproducible manner.

In accordance with one embodiment, methods are provided for reconstituting in vitro the original in vivo tissue microenvironment present in native primary tissues (including microenvironments as present in primary tumor, tumor metastasis, tumor draining lymph nodes, or pleural effusions) after the tissue has been dissociated into individual cells. In accordance with one embodiment the primary tissue is first dissociated into individual cells that are suspended in a liquid or semi-solid extracellular matrix/hydrogel, and then aggregated into uniform three dimensional clusters of cells by the application of an aggregating force, wherein the clusters of cells comprise the representative cell types present in the original primary tissue.

In one embodiment, a method of preparing reconstituted 3D clusters is provided. The method comprises
dissociating a tissue recovered from a subject into a mixture of dissociated individual cell types;
mixing the dissociated individual cells in a liquid medium;
manipulating the mixture to aggregate the dissociated individual cell types into reconstituted 3D clusters, wherein each reconstituted 3D cluster comprises multiple cell types and the reconstituted 3D clusters comprises a microenvironment that mimics functionality, cell attachment and cell to cell interaction of the tissue. In one embodiment, manipulating the mixture of dissociated individual cell types comprises applying an acoustic force or a centrifugation force sufficient to aggregate the dissociated individual cell types into discreet 3D clusters of cells.

In one embodiment, the method disclosed herein is used to prepare 3D clusters of cells derived from a primary tumor, wherein the 3D cell clusters comprise tumor cells, myeloid derived suppressor cells (MDSCs), and T-cells. Once the dissociated cells from a recovered tissue sample have been aggregated into clusters comprising the different cell types of the original tissue, the aggregating force can be removed and the clustered cells can be stabilized (i.e. cells of the cluster held in communication contact with each other) using standard techniques including for example by gelling or inducing the formation of a matrix in the liquid media of the cell suspension. Accordingly, in one embodiment the 3D cell clusters are embedded within a matrix. More particularly, in one embodiment the liquid medium comprises a polymerizable component, and the method may comprise a step of stabilizing the 3D cell clusters comprises by inducing the liquid medium to form a matrix after the cells are aggregated into 3D cell clusters. In one embodiment the liquid medium is induced to form a matrix by an alteration in temperature, pH, or by exposure to UV light.

In accordance with one embodiment, the 3D clusters of cells formed in accordance with the present disclosure may be used to study cellular interactions and investigate the effects of the removal or addition of various cell types or the addition or subtraction of various bioactive agents or environmental factors. Such alterations can be introduced during the process of aggregating the dissociated cells into clusters or after the clusters have been formed. In one embodiment, prior to, or during the aggregation step of the dissociated cells, the mixture of dissociated individual cell types is subject to alteration or manipulation, wherein the alteration or manipulation comprises:
i) removing one or more specific cell types from the mixture;
ii) altering the ratio of the cell types present in the mixture;
iii) adding a bioactive agent to the mixture, wherein the bioactive agent is DNA, RNA protein or living organism (prokaryotic or eukaryotic cells) that has a biological effect on cells, optionally wherein the bioactive agent is a pharmaceutical, optionally an anti-tumor agent;
iv) adding cells (prokaryotic or eukaryotic cells) to said mixture, optionally wherein the added cells represent a component cell of the original tissue, or another cell type from the host that was the source of the original tissue, or an cell exogenous to the host that was the source of the original tissue, or cells autologous from patient peripheral blood or tumor tissues, either unmodified or genetically engineered such as autologous chimeric antigen receptor T cells (CAR-T) or allogenic cell therapy products, such as allogenic CAR-T or CAR-NK cells;
v) exposing the cells to an environmental manipulation, for example a change in temperature, pH, or irradiation) or
vi) any combination of two or more of i), ii), iii) and iv).

In accordance with one embodiment of the present disclosure, an array of assembled three dimensional (3D) cell clusters, prepared from dissociated cells of a primary tissues of a subject, is provided using the methods disclosed herein. In one embodiment the 3D cell clusters comprise multiple cell types of the original primary tissue. In some embodiments, each 3D cluster comprises multiple cell types and generates a microenvironment that mimics the functionality, cell attachment, and cell to cell interaction of the original tissue.

In one embodiment the 3D cell clusters are formed from the dissociated cells of a primary tumor recovered from a subject, wherein the clusters comprise tumor cells, myeloid derived suppressor cells, and T-cells. In one embodiment the array of assembled 3D cell clusters comprises a plurality of cell clusters of uniform composition that are suspended, optionally at uniform distance from one another, in a scaffold structure, optionally wherein the scaffold is a matrix of polymers. In one embodiment the scaffold structure is formed by polymerizing subunits present in the original media comprising the cells.

In one embodiment the 3D cell clusters disclosed herein are prepared from dissociated cells of primary tissues through the use of an acoustic force, using standard devices known to those skilled in the art to quickly aggregate dissociated primary tissues/tumor cells into 3D clusters. The clusters consist of original tissue/tumor components with the option to substitute/addition/removal of one or more components to interrogate its/their roles in tissue/tumors' growth, function and responses to treatments.

In one embodiment the primary tissue that is dissociated into individual cells is primary tumor tissue recovered from a patient. Briefly, tissues/tumors are dissociated into single cell suspension. At this stage, cell components could be modified, substituted, removed, or added. The unmodified and/or modified cell suspensions will then be quickly aggregated into 3D clusters though acoustic devices. The aggregated cells can then be interrogated by time-lapse imaging, qPCR, ELISA, flow cytometry, RNA sequencing, electrochemical measurement such as meso scale discovery assays (MSD), or other molecular/cellular assays to assess cell-cell interactions and responses to treatments.

In accordance with one embodiment, a method of identifying cancer immunotherapy drugs comprises assembling an array of three dimensional (3D) cell clusters prepared from dissociated cells of a tissue of a subject, and evaluating the function and response of one or more drugs in the array of three dimensional (3D) cell clusters, wherein acoustic waves are used to form the array of 3D cell clusters.

The present disclosed compositions and methods provide a novel method/model to study interactions of original tissue/tumor components ex vivo by fast aggregation of dissociated single cells into 3D organotypic arrays. The organotypic tissue/tumor cultures can serve as (1) a model to study biological functions, (2) a companion diagnostic assay for cancer/disease prognosis, treatment selection, and personalized therapy, and (3) a model to screen for novel therapeutics.

### BRIEF DESCRIPTION OF THE DRAWINGS

FIGS. 1A-ID: FIG. 1A: Concept of acoustically assembled patient-derived cell clusters (APCCs) for MDSCs targeting drug studies. FIG. 1B: The simulation of acoustic fields. FIG. 1C: The 3D cell patterning in Matrigel using acoustic assembly. FIG. 1D: The temperature dynamics when applying acoustic signal (grey region). Scale bar: 1 mm.

FIGS. 2A-2D. Acoustically assembled patient-derived cell clusters (APCCs) vs 2D cultures. FIG. 2A: Enumeration of CD15+ cells and CD4/8+ T cells within each APCC clusters. FIG. 2B Quantification of tumor cells, CD15+ cells and CD4/8+ T cells' viability within the APCCs and comparison with 2D culture conditions, n=3. FIG. 2C: ELISA analysis of TNF-α and IFN-γ concentrations in culture supernatants from the 2D and APCC cultures, n=3. FIG. 2D: Gene expression analysis of CD15+ cells and CD4/8+ T cells in the 2D/APCC conditions from dissociated tumors, at 0 hour, and at 24 hours post acoustic assembly, n=3.

FIGS. 3A-3B: T cell cytotoxicity suppression by myeloid derived suppressor cells (MDSCs). Acoustically assembled patient-derived cell clusters (APCCs) were prepared i) with all tumor components (control), ii) with CD15+ cells removal and iii) with CD15+ cells plus CD4/8+ T cells co-removal conditions. FIG. 3A presents data from tumor cell death quantification within the APCCs in each of the prepared sets of APCCs: control, CD15+ cells removal and CD15+ cells plus CD4/8+ T cells co-removal conditions, n=3. FIG. 3B: ELISA analysis of TNF-α and IFN-γ concentrations in culture supernatants from control, CD15+ cells removal and CD15+ cells plus CD4/8+ T cells co-removal conditions, n=3.

FIGS. 4A-4C: Evaluation of myeloid derived suppressor cells (MDSCs) targeting therapy using acoustically assembled patient-derived cell clusters (APCCs). APCCs formed in accordance with the present disclosure with treated with, i) anti-PD1, ii) anti-PD1 in combination with Cabozantinib and iii) anti-PD1I treatment with MDSCs removal conditions or left untreated (control). FIG. 4A presents data showing tumor cell death quantification within the APCCs treated with anti-PD1, anti-PD1 in combination with Cabozantinib and anti-PD1 treatment with MDSCs removal conditions, or left untreated (control) n=3. FIG. 4B shows ELISA analysis of TNF-α and IFN-γ concentrations in culture supernatants from control, anti-PD1, anti-PD1 in combination with Cabozantinib and anti-PD1 treatment with MDSCs removal conditions, n=3. FIG. 4C presents data from qRT-PCR analysis of purified MDSCs and T cells isolated from APCCs 24 hours post-treatment under control, anti-PD1, anti-PD1 in combination with Cabozantinib and anti-PD1 treatment with MDSCs removal conditions, n=3.

FIGS. 5A-5G: Optimization of acoustic field for patient-derived cells assembly. FIG. 5A illustrates schematics showing configuration of acoustic devices. FIG. 5B illustrates schematics of acoustic field inside cell culture chamber. FIG. 5C illustrates simulation and results of acoustic fields assembling polystyrene (PS) beads with single, pairs of piezoelectric transducers (PZT) or both pairs of PZT turned on. FIG. 5D shows a distribution of PS beads along the X and Y axis of acoustic fields. FIG. 5E shows a distribution of distances between neighboring acoustic pressure antinodes. FIG. 5F shows varying sizes of cell clusters can be formed by acoustic assembly input cell concentrations were changed. FIG. 5G shows quantification of cell cluster sizes with corresponding input cell concentrations. Scale bar: 1 mm.

FIGS. 6A-6B: Immune cell profiling of tumor microenvironment (TME) components of dissociated EO771 mouse primary tumor and acoustically assembled cell clusters (APCCs). FIG. 6A shows a gating strategy to analyze TME immune cell components. FIG. 6B shows a comparison of T cell and myeloid derived suppressor cells (MDSCs) makeup in dissociated primary tumor cells and APCCs. Cell components percentage are calculated as corresponding cell count/total viable cell count.

### DETAILED DESCRIPTION

### Definitions

In describing and claiming the methods, the following terminology will be used in accordance with the definitions set forth below.

The term “about” as used herein means greater or lesser than the value or range of values stated by 10 percent, but is not intended to designate any value or range of values to only this broader definition. Each value or range of values preceded by the term “about” is also intended to encompass the embodiment of the stated absolute value or range of values.

As used herein, the term “treating” includes alleviation of the symptoms associated with a specific disorder or condition and/or preventing or eliminating said symptoms.

As used herein the terms “effective amount” or “therapeutically effective amount” of a compound refers to a nontoxic but sufficient amount of the compound to provide the desired effect. The amount that is “effective” will vary from subject to subject, depending on the age and general condition of the individual, mode of administration, and the like. Thus, it is not always possible to specify an exact “effective amount.” However, an appropriate “effective” amount in any individual case may be determined by one of ordinary skill in the art using routine experimentation.

The term “parenteral” means not through the alimentary canal but by some other route such as intranasal, inhalation, subcutaneous, intramuscular, intraspinal, or intravenous.

As used herein, the term “purified” relates to the isolation of a molecule or compound in a form that is substantially free of contaminants normally associated with the molecule or compound in a native or natural environment and means having been increased in purity as a result of being separate from other components of the original composition.

As used herein the term “subject” means an animal including but not limited to, humans, domesticated animals including horses, dogs, cats, cattle, and the like, rodents, reptiles, and amphibians

As used herein the term “patient” means an animal including but not limited to, humans, domesticated animals including horses, dogs, cats, cattle, and the like, rodents, reptiles, and amphibians being administered a therapeutic treatment either with or without physician oversight.

As used herein the term “bioactive agent” includes any moiety that has a biological effect on cell or organism including effects on viability, metabolism, or any cell function. In accordance with one embodiment the bioactive agent can be a DNA, RNA, protein, carbohydrate, cell matrix component or a cell or cell fragment.

Solid tumors, as well as other solid tissues often host a tissue-specific milieu that reprograms infiltrating immune cells to induce a tissue specific phenotype, such as mucosal associated invariant T cells (MAITs), brain resident meningeal macrophages (MGMs), and tumor induced myeloid derived suppressor cells (MDSCs). Once taken out of the milieu, the in situ phenotypes of these tissue resident immune cells are often quickly lost due to diluted paracrine cytokine environment and lack of cell-cell interactions. In accordance with one embodiment of the present disclosure, the acoustically assembled patient-derived cell cluster (APCC) that can quickly reconstitute the local cell-cell interactions and/or paracrine factors in a fast, high-throughput and label-free manner is described. In some embodiments, the cells may be aggregated into compact, uniform clusters that preserve the original tumor compositions by applying bio-compatible bulk acoustic waves onto dissociated tumor cell suspensions

It is difficult to culture TME niche cell types including tumor induced MDSCs. MDSC is a key immune suppressive cell type within the TME that mediates resistance to ICI treatments. There has been growing interest to target MDSCs in ICI resistant or refractory solid tumors. Additionally, MDSCs have also been shown to play key roles in non-tumor settings such as acute bacterial and viral infection, as well as autoimmune diseases. However, traditional 2D cultures of isolated MDSCs will lead to rapid cell death as well as loss of key phenotypes.

Tumor immunity mediates tumor initiation, progression, and response to treatment through the dynamic and complex crosstalk among multiple tumor and immune cells with tumor immune microenvironment niche phenotypes. However, current patient-derived models such as tumor organoids and 2D cultures lack some essential niche cell types (e.g., myeloid derived suppressor cells or MDSCs) and fail to model complex tumor-immune interactions, limiting their potential in recapitulating tumor immunity of an individual cancer patient. The acoustically assembled patient-derived cell clusters (APCC) described in the present disclosure are advantageous because they preserve viability and functional phenotypes of a sensitive microenvironment.

In accordance with one embodiment of the present disclosure, a novel model comprising of acoustically assembled patient-derived cell clusters (APCC) that can preserve original tumor and immune cell compositions is described. In accordance with one embodiment of the present disclosure, the interactions of the acoustically assembled patient-derived cell cluster (APCC) in 3D microenvironments is modelled. In accordance with one embodiment of the present disclosure, responses of the acoustically assembled patient-derived cell cluster (APCC) to primary patient tumor treatments are predicted in a rapid, scalable, and/or user-friendly manner.

In accordance with one embodiment of the present disclosure, by incorporating a large array of 3D acoustic trappings, hundreds of APCCs can be assembled within a petri dish or any other container from cell suspensions derived from fresh primary patient tumor tissues within minutes. In accordance with one embodiment of the present disclosure, the APCCs may preserve sensitive and short-lived (˜1 to 2-day lifespan in vivo) tumor induced MDSCs, In accordance with one embodiment of the present disclosure, the APCCs may exhibit MDSC suppression dynamics of T cells mediated tumor cell toxicity up to about 24 hours. The MDSC suppression was confirmed by using time-lapse live-cell imaging and biochemical assays.

As described in the present disclosure, fast (about 2 minutes), and label-free acoustic assembly of the APCCs preserves the viability of MDSCs. In some embodiments, assembly of the APCCs may occur in about 1 minute to about 5 minutes, about 5 minutes to about 10 minutes, about 10 minutes to about 20 minutes, including any time or range comprised therein. In some embodiments, the phenotypes including expression of arginase and NO productions related genes, which were otherwise quickly lost in 2D culture are also preserved. In accordance with one embodiment of the present disclosure, the MDSCs inside these APCCs may also function to inhibit T cell mediated tumor cell death in vitro.

Patient derived tissues/cells hold important genetic, epigenetic, transcriptomic, and proteomic information unique to the patient genetics and treatments they received. Profiling of patient derived tissue has shown promising potentials for disease diagnosis, prognosis, and precision medicine. Traditional molecular profiling technologies such as DNA/RNA sequencing treat tumor as a bulk tissue and lack information regarding functional phenotypes of specific cell types within the tissues. Single cell profiling technologies such as CyTOF or single cell sequencing could provide information regarding subpopulations of tissue cells, but they are rather expensive and cannot be used to study drug treatment responses directly. Xenografting tumor fragments into immune-compromised animals can be used for individualized drug treatment studies but they require large quantity of tissues and not amendable to high throughput screening studies. They also cannot be used to study immune components within the original tissue. In accordance with one embodiment of the present disclosure, the APCCs may serve as a model or platform for testing the effect of existing drugs on MDSC functions. Such testing may be used for identifying drugs that can be potentially utilized for cancer immunotherapy.

Many previously FDA approved drugs were initially intended for other applications/indications. For example, Cabozantinib was initially approved to treat advanced renal carcinoma for its anti-angiogenesis activity. As demonstrated in the present disclosure, and known previously, Cabozantinib may also function through inhibition of PMN-MDSC activity. Thus, the present disclosure can be used to screen for FDA approved drugs to elucidate their TME modulation functions for novel drug applications and discoveries. In accordance with one embodiment of the present disclosure, the APCCs may preserve cellular and functional profiles of sensitive immune cell populations such MDSCs within the TME.

In accordance with one embodiment of the present disclosure, the APCCs may successfully exhibit combinational therapeutic effect of a multi-kinase inhibitor targeting MDSCs (Cabozantinib) and/or a PD-1 immune checkpoint inhibitor (Pembrolizumab) to promote antitumor immunity. In accordance with one embodiment of the present disclosure, the APCCs may predict responses of an individual patient to different cancer therapies ex vivo. In accordance with one embodiment of the present disclosure, the APCCs may be utilized to screen novel cancer immunotherapy and combinational therapy.

In accordance with one embodiment of the present disclosure, the APCC may provide a platform where original tissue make-up could be preserved in acoustic assembled 3D cultures with rapid profiling of drug treatment responses with data readout within 24 hours. The APCC may be used for MDSC studies to explore their biological functions ex vivo utilizing patient derived cells. The APCC may be used for applications that require preservation of localized, complex cell-cell communications, such as modeling lymph node immunity and inflammatory hot-spots known as tertiary lymphoid structures (TLS) in various organs.

In accordance with one embodiment of the present disclosure, the APCC is a label-free, cell-type agnostic tool that may be used to study localized cell-cell paracrine signaling and interactions in a standardized, uniform, and/or scalable manner. In accordance with one embodiment of the present disclosure, the APCC has broad potential for immunotherapy screening applications as well as utility as a companion diagnostics for precision medicine. Tumor cells and tumor associated immune cell phenotypes can quickly change their phenotypes after dissociation in to individual cell types.

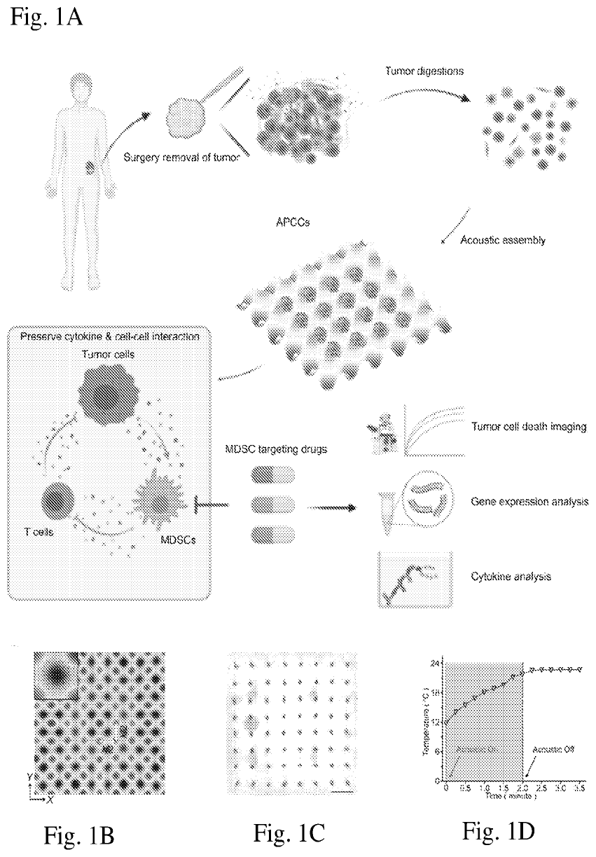

As shown in FIG. 1A, fast assembly of tumor cells post dissociation into 3D cell clusters is critical to preserve the original tumor components and functional phenotypes. In accordance with one embodiment of the present disclosure, acoustic devices with 2 pairs of piezoelectric transducers (PZTs), generating biocompatible bulk acoustic waves were designed to achieve fast, scalable, and size uniform acoustically assembled patient-derived cell clusters (APCCs). The center chamber was inserted with disposable, standard 30 mm cell culture petri-dishes holding the dissociated tumor cells. Once acoustics were applied, cell suspensions were quickly aggregated into typically about 100 acoustic pressure nodes after about 2 minutes of acoustics application, forming an uniform array of APCCs as shown in FIG. 1B, 1C. In other embodiments, the number of acoustic pressure nodes may range from about 50 to about 1000, including any number or range comprised therein.

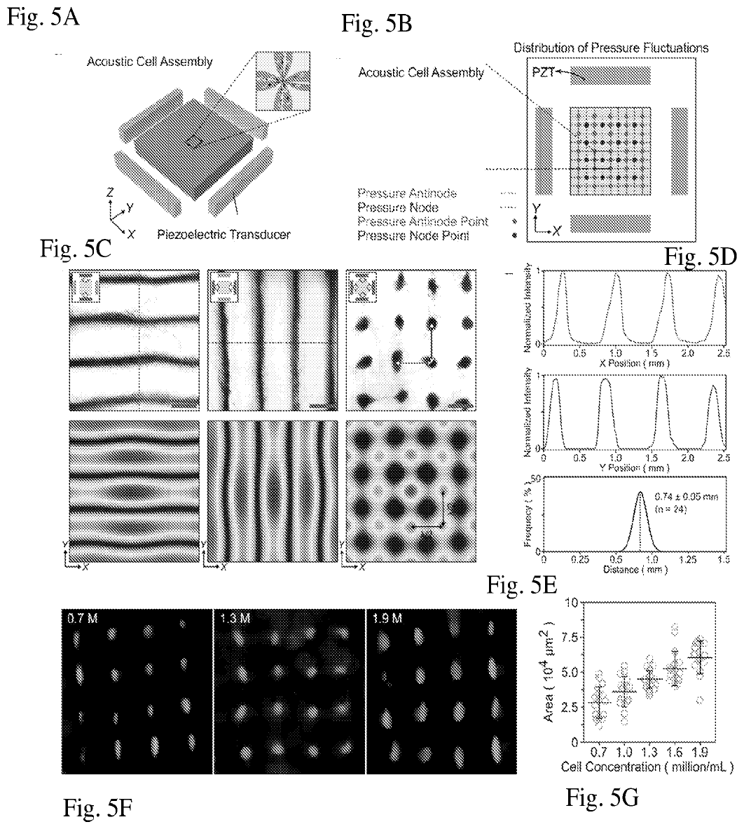

In one embodiment, cell clusters were then held in place by Matrigel as Matrigel gelates at room temperature during the assembly process (FIG. 1D). The diameters of the initial cell clusters may range from 150 μm to 250 μm, including any diameter or range of diameter comprised therein. The cell clusters may comprise an input cell concentrations ranging from about 0.7 million/mL to 1.9 million/mL, including any cell concentration or range of cell concentration comprised therein (FIG. 5A-5G).

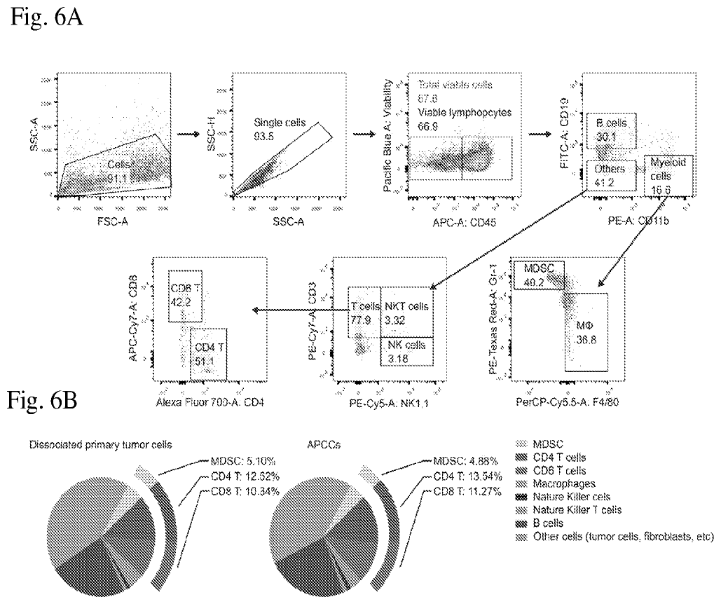

In one embodiment, the cell suspensions were seeded at a concentration of about 1.5 million cells per milliliter, which yields about 2,500 cells per APCCs. Using dissociated mouse syngeneic EO771 primary breast tumor as controls, it was confirmed that the APCCs could preserve the makeup of the original tumor microenvironment immune cells. Additionally, it was shown that the original T cells and MDSCs percentages were preserved (FIG. 6A-6B).

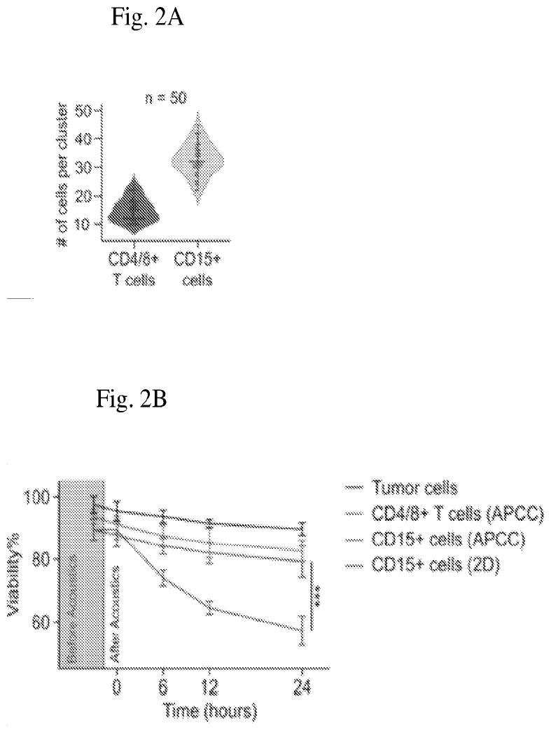

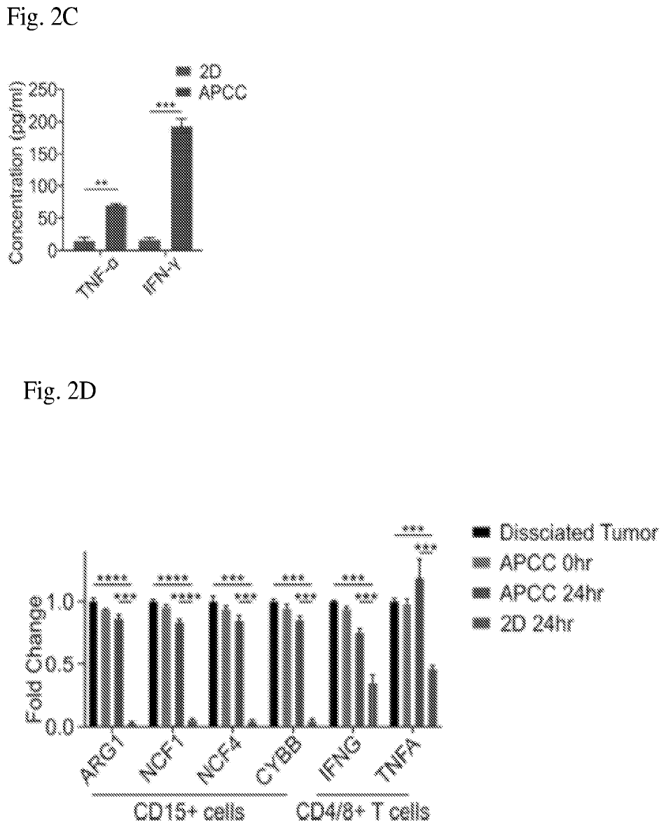

To evaluate the preservation of MDSC viability inside APCCs, initial tests were performed with renal cell carcinoma (RCC) primary tumors from patients. For MDSC study, the CD15+ cells were first isolated from dissociated patient tumor cells via magnetic microbeads, which consist of tumor infiltrating neutrophils including immunosuppressive polymorphonuclear myeloid derived suppressor cells (PMN-MDSCs). The CD15+ cells were pre-labelled with DiL membrane dyes (shown as yellow). T cells were isolated via a 1:1 mixture of CD4 and CD8 microbeads and pre-labeled the T cells with DiO membrane dyes. The CD15+, CD4+, CD8+ depleted dissociated tumor cells were labeled with the blue CMAC cell tracker dye. Membrane impermeable cell nucleus staining NucRed dead 647 was added to indicate cell death. The cell death of the CD15+ cells was calculated by analyzing co-localization of Dil labeled MDSCs and NucRed cell death indicator dye over time-lapse imaging (FIG. 2A).

In accordance with one embodiment of the present disclosure, the distribution of the CD15+ cells and CD4/CD8+ T cells across 50 APCCs was analyzed. CD15+ cells and CD4/CD8+ T cells showed relatively uniform distribution across these APCCs, with an average of 15±4 CD4/CD8+ T cells and 32±5 CD15+ cells within each APCC (FIG. 2B). The viability of CD15+ cells maintained in the 2D culture and in APCCs were compared to illustrate that CD15+ cells maintained in the 2D dissociated tumor culture showed significant cell death over the 24-hour in vitro culture time. The viability decreased significantly from 89.5%±3.4% at 0 hours to 57.2%±4.7% at 24 hours. In contrast, the CD15+ cells inside APCCs showed minimal cell death upon acoustics treatment (before treatment viability: 89.5%±3.4% versus after treatment viability: 87.8±3.7%) and remained highly viable over 24 hours culture period (24-hour viability: 79.3±5%).

In accordance with one embodiment of the present disclosure, the cytokine secretion profiles from both the 2D cultures and APCC cultures were analyzed. The APCC culture supernatants showed a 12-fold increase in IFN-γ and 4.8-fold increase in TNF-α concentrations as compared with 2D cultures (FIG. 2C), demonstrating the capacity of APCC cultures to better preserve local cytokine microenvironment.

In accordance with one embodiment of the present disclosure, the CD15+ cells and CD4/CD8+ T cells were isolated at 24 hours from APCCs and analyzed the expression of key immune-suppressive genes of MDSCs (ARG1, NCF1, NCF4, CYBB) which mediates MDSC arginase 1 secretion and NO generation to inhibit T cells within tumor TME. In one embodiment, ARG1, NCF1, NCF4, and CYBB expression were quickly lost in the CD15+ cells in 2D cultures. This may be due to the lack of cell-cell contacts. However, these gene signatures were unaltered by acoustic assembly and largely preserved over the 24 hours culture period in the APCCs. Similarly, T cells' expressions of TNF-α and IFN-γ were also better preserved by the APCCs as compared with 2D culture conditions (FIG. 2D).

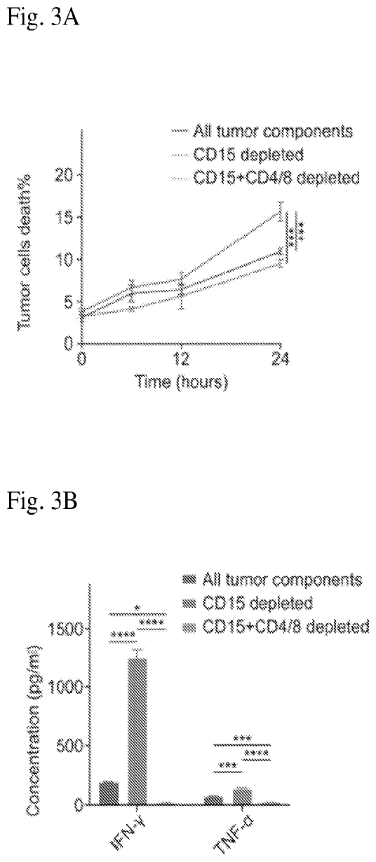

One of the main functions of MDSCs in tumor is that they inhibit T cell mediated tumor cell killing. To investigate whether this function is preserved in the APCCs, in accordance with one embodiment of the present disclosure, the effect of removal of MDSCs on cell-interactions within APCCs was evaluated. The CD15+ cells (including PMN-MDSCs) were pre-removed from dissociated tumor cells by magnetic based depletion. Tumor cell death was observed by time-lapse imaging within the control and in APCCs without CD15+ cells. In one embodiment, upon CD15+ cells removal, tumor cell death within the APCCs was increased by about 43.4%±10.8% at 24 hours (FIG. 3A). To further confirm that this enhanced tumor cell death was T cell dependent, the CD4/8+ T cells and the CD15+ cells were co-removed from APCCs. In one embodiment, after the co-removal of CD4/8+ T and CD15+ cells, the tumor cell death was significantly reduced to a level even lower than the control conditions over time.

In accordance with one embodiment of the present disclosure, the cytokine microenvironment change upon the removal of CD15+ cells was evaluated. In one embodiment, CD15+ cells removal enhanced TNF-α and IFN-γ cytokine levels within the APCCs, as an indicator of potential enhanced T cell activation. Furthermore, this increased cytokine secretion was negated upon T cell co-removal (FIG. 3B). Thus, in one embodiment, the CD15+ cells inside APCCs may have preserved MDSC function to inhibit T cells.

MDSC-mediated immune evasion is a key mechanism underlying tumor development. Thus, there is a growing interest to develop drugs targeting MDSCs to overcome ICI resistance. In accordance with one embodiment of the present disclosure, the APCCs were validated as a potential model or platform to study therapeutic effects of

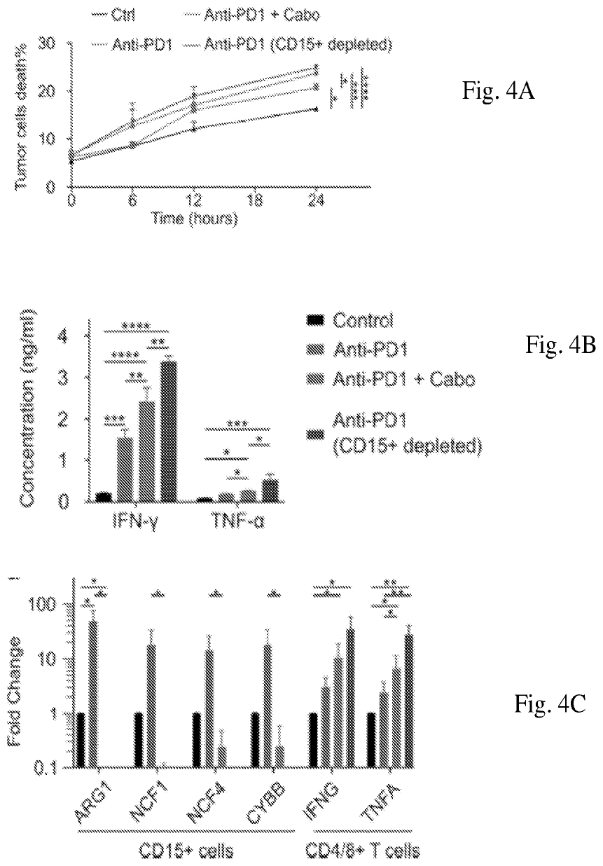

MDSC-targeting drugs alone and/or in combination with ICIs. The APCCs derived from 3 renal cell carcinoma (RCC) primary tumors of patients were treated with immune checkpoint inhibitor anti-PD1 (Pembrolizumab) and/or a MDSC-targeting drug Cabozantinib. The tumor cell death over a 24-hour time lapse was observed. In one embodiment, anti-PD1 treatment alone could enhance the tumor cell death by about 17.0%±9.4% inside RCC derived APCCs as compared to untreated controls. In one embodiment, when anti-PD1 and Cabozantinib treatments were combined, the tumor cell death within the APCCs was increased by about 33.7%±6.5% as compared to the untreated control. Thus, a 123%±67% increase in drug induced tumor cell death was observed in the combined treatment as compared with the anti-PD1 treatment alone. In one embodiment, the effect of anti-PD1 treatments on APCCs without CD15+ cells was evaluated. This group yielded an even higher tumor cell death with an increase of about 40.9%±9.4% as compared the untreated control group (FIG. 4A).

In one embodiment, the cytokine levels of TNF-α and IFN-γ were evaluated in APCCs treated with anti-PD1, in APCCs treated with an anti-PD1 and Cabozantinib combination, and in APCCs without with CD15+ cells that were treated with anti-PD1. The levels of TNF-α and IFN-γ were elevated in the APCCs treated with anti-PD1. The levels of TNF-α and IFN-γ were further elevated in APCCs treated with an anti-PD1 and Cabozantinib combination and in the without with CD15+ cells that were treated with anti-PD1. (FIG. 4B).

In one embodiment, the MDSC suppressive gene expression in CD15+ cells isolated from APCCs treated with anti-PD1 and from APCCs treated with an anti-PD1 and Cabozantinib combination was evaluated. The MDSCs suppressive genes were slightly upregulated in APCCs treated with anti-PD1. This upregulation may be in response to enhanced T cell activity. But these suppressive gene expressions were almost completely absent in CD15+ cells from APCCs treated with an anti-PD1 and Cabozantinib combination, suggesting Cabozantinib effectively inhibited MDSCs' suppressive function. (FIG. 4C).

In one embodiment, TNF-α and IFN-γ expression in CD4/CD8+ T cells isolated from APCCs treated with anti-PD1, from APCCs treated with an anti-PD1 and Cabozantinib, and from APCCs without with CD15+ cells that were treated with anti-PD1 was evaluated. In accordance with ELISA data, TNF-α and IFN-γ gene expressions were enhanced by anti-PD1 treatment, which were further augmented by combinational therapy with Cabozantinib. The TNF-α and IFN-γ gene expression was the highest in APCCs without with CD15+ cells that were treated with anti-PD1. (FIG. 4C).

### Example 1: Preparing Acoustically Assembled Patient-Derived Cell Clusters

The acoustic platform was comprised of an inner PMMA chamber of 40×40 mm to hold a 30 mm diameter sterile cell culture petri-dish and four flanking slots of 20×5 mm to hold 2 opposing piezoelectric transducer pairs. The 2 piezoelectric transducer pairs have slightly different resonant frequencies, which was 0.996 MHz and 1.006 MHz respectively.

Fresh patient renal cell carcinoma (RCC) tumors were collected by the tissue procurement and distribution core, Indiana University Melvin and Bren Simon Comprehensive Cancer Center under IRB protocol #1907977109. Tissues were weighted and digested using human tumor dissociation kit using a gentle MACS dissociator (Miltenyi).

For acoustic cell assembly, a sterile 30 mm diameter petri-dish holding 200 μL of cell suspensions was inserted into the inner PMMA chamber. Sterile deionized water was then carefully added to the space between the petri-dish and PMMA chamber to conduct acoustics. To assemble cell suspensions into APCCs, 200 μL of patient primary tumor derived single cell suspensions in Marigel (Corning) (37.5% v/v in RPMI-1640 culture medium, Gibco) was added to the sterile petri dish. Signal inputs (0.996 MHz and 1.006 MHz) with 20% duty cycle and 200 m Vpp were applied to two pair transducers, respectively. After two minutes, cells were aggregated into clusters with uniform sizes by the acoustic radiation force. Meanwhile, the temperature was raised to 24° C., leading to gelation of the Matrigel, fixing the cell clusters in place. The acoustics were then turned off and the petri-dish was left on stage for 30 minutes to allow the Matrigel to fully gelate. The cells in the petri-dish were then topped with 100 μL of pre-warmed complete medium (RPMI-1640+10% fetal bovine serum+1X MEM Non-Essential Amino Acids supplement+1X GlutaMAX, Gibco) and transferred into a 37° C. on-stage incubation chamber (Tokai-hit) or standard cell culture incubator for continuous culture.

### Example 2: Isolation and Labeling of Tumor Immune Components

Digested tumor cells were equally divided into 3 tubes. The CD15+ cells in the first tube are labelled with CD15 magnetic beads (Miltenyi) and the cells are isolates by positive selection. The T cells in the second tube are labeled with CD4 and CD8 magnetic beads (1:1) (Miltenyi) and the cells are isolated by positive selection. All three markers in the third tube are labeled with magnetic beads (CD15, CD4, and CD8) and CD15+ cells and CD4/8+ T cells are depilated by negative selection. This results in CD15+ and CD4/8+ T cells co-depleted tumor components. The T cells and CD15+ cells were re-suspended in RPMI-1640 medium (serum-free) at 1 million cells per 1 mL to stain with Vybrant DiO and DiL (Invitrogen) at 1:1,000 dilutions at 37° C. for 1 hour. T cells and CD15+ cells co-depleted tumor components were re-suspended in complete RPMI-1640 medium at 1 million cells per 1 mL and stained with CellTracker Blue CMAC dye (Invitrogen) at 1 μM at 37° C. for 1 hour. Labeled CD4/8+ T cells, CD15+ cells, and CD4/8+CD15 co-depleted tumor components were then washed twice in complete RPMI-1640 medium and mixed back. The cell mixtures were then re-suspended at 1.5 million cells per milliliter in 37.5% Marigel in pre-chilled complete RPMI-1640 medium (v/v) to prepare for acoustic assembly. Before acoustic assembly, 1 drop of NucRed Dead 647 ReadyProbes (Invitrogen) was added to 1 milliter of cell suspensions and left in medium to visualize cell death over time.

### Example 3: Flow Cytometry Analysis of Mouse Primary Tumor and APCCs

Orthotopic mouse tumors were digested using mouse tumor dissociation kit (Miltenyi). Digested single cell suspensions were re-suspended in flow buffer made with filtered 1X phosphate buffered saline (PBS, Gibco), supplemented with 10% fetal bovine serum (FBS, Gibco). Cell suspensions were then assembled into APCCs. For flow cytometry labeling, cells were then retrieved from APCCs by gentle digestion with cell recovery solution (Corning) and re-suspended in flow buffer. The cells were then labeled with corresponding fluorophore conjugated antibodies (Table 1) for 30 minutes at 4 degrees. The labeled cells were washed twice with flow buffer and analyzed using a BD LSRII flow cytometer. Compensation controls were prepared using anti-rat or anti-mouse compensation particles (BD) and run together with the samples.

TABLE 1
Antibody used in flow cytometry analysis
of mouse EO771 primary tumors

| Antigen | Fluorophore | Host | Vendor | Catalog# | Dilution |
|---|---|---|---|---|---|
| CD45 | APC | Rat | Biolegend | 103111 | 1:100 |
| CD3 | PE/Cy7 | Rat | Biolegend | 100219 | 1:200 |
| CD4 | Alexa Fluor 700 | Rat | Biolegend | 100429 | 1:200 |
| CD8a | APC/Cy7 | Rat | Biolegend | 100713 | 1:200 |
| CD19 | FITC | Rat | Biolegend | 152403 | 1:100 |
| CD11b | PE | Rat | Biolegend | 101207 | 1:200 |
| F4/80 | PerCP/Cy5.5 | Rat | Biolegend | 123128 | 1:200 |
| NK1.1 | PE/Cy5 | Mouse | Biolegend | 108715 | 1:100 |
| Gr-1 | PE-eFluor 610 | Rat | Invitrogen | 61-5931-82 | 1:100 |

### Example 4: Drug Treatment of the APCCs

For drug treatments, anti-PD1 antibodies (Pembrolizumab, SelleckChem) was added at 5 μg/mL to the APCCs. Cabozantinib (SelleckChem) was added at 10 μM to the APCCs.

### Example 5: qRT-PCR Analysis of the MDSCs and T Cells

For qRT-PCR analysis of the CD15+ cells and CD4/8+ T cells, CD15+ cells or CD4/8+ T cells were pre-labeled by CD15 or CD4-and-CD8 (1:1) magnetic beads for 15 minutes at 4° C. respectively before mixed with other tumor cells for acoustic assembly. The CD15+ cells or CD4/8+ T cells with the labeling beads were then assembled into APCCs. To analyze their gene expression profiles, the APCCs were digested by with cell recovery solution (Corning), and input onto MACS columns (Miltenyi) for positive magnetic separation to isolate pure CD15+ cells or T cells (CD4+ or CD8+). The purified CD15+ cells or CD4/8+ T cells were then lysed for RNA extraction by RNeasy Mini Kit (Qiagen), reverse transcribed to cDNA by High-Capacity cDNA Reverse Transcription Kit (Applied Biosystems), and analyzed using corresponding primer pairs (Table 2) and SYBR Green PCR Master Mix (Applied Biosystems). The samples were run on a StepOne Real Time PCR machine (Applied Biosystems) to quantify signals. The results were analyzed using AACt method for fold change analysis.

### Sequences

Provided herein is a representative list of certain sequences included in embodiments provided herein.

TABLE 2
Primer sequences for qPCR analysis

| SEQ ID NO: | Gene | Primer sequence |
|---|---|---|
| 1 | hGAPDH_Fwd | AGGTCGGAGTCAACGGATTT |
| 2 | hGAPDH_Rev | TTCCCGTTCTCAGCCTTGAC |
| 3 | hArg1_Fwd | ACGGAAGAATCAGCCTGGTG |
| 3 | hArg1_Rev | GGCACATCGGGAATCTTTCCT |
| 4 | hNCF1_Fwd | GAGTACCGCGACAGACATCA |
| 5 | hNCF1_Rev | CGCTCTCGCTCTTCTCTACG |
| 6 | hNCF4_Fwd | AGAAGAGAGGCTTCACCAGCCA |
| 7 | hNCF4_Rev | TCCTCCAGCTTGCTCTGCAAAG |
| 8 | hCYBB_Fwd | CTCTGAACTTGGAGACAGGCAAA |
| 9 | hCYBB_Rev | CACAGCGTGATGACAACTCCAG |
| 10 | hTNFA_Fwd | CTCTTCTGCCTGCTGCACTTTG |
| 11 | hTNFA_Rev | ATGGGCTACAGGCTTGTCACTC |
| 12 | hIFNG_Fwd | GAGTGTGGAGACCATCAAGGAAG |
| 13 | hIFNG_Rev | TGCTTTGCGTTGGACATTCAAGTC |

### Example 6: ELISA Analysis of Cytokines

To perform cytokine analysis on the APCCs and 2D tumor cultures, 50 μL supernatants were pipetted out of the culture vessel and centrifuged at 10,000 g for 10 minutes to remove cell debris. The supernatants were then analyzed by human TNF-α or IFN-γ kits (Tribioscience).

### Example 7: Statistical Analysis

All data were extracted and analyzed using Prism 7 (GraphPad Software). P-value between 2 samples were analyzed by student's t-tests. P-value among 3 or more samples were analyzed by one-way ANOVA followed by Tukey's honestly significant difference (HSD) post hoc test. P-values were denoted as following: *<0.05; **<0.01; ***<0.005; ****<0.001.

TABLE 3
Patient Demographics

| # | Diagnosis | Age | Race | Sex | Surgery |
|---|---|---|---|---|---|
| Patient 001 | Renal cell | 74 | Caucasian | Male | Right |
| | carcinoma | | | | nephrectomy |
| Patient 002 | Renal cell | 64 | Caucasian | Male | Left |
| | carcinoma | | | | nephrectomy |
| Patient 003 | Renal cell | 52 | Caucasian | Male | Right |
| | carcinoma | | | | nephrectomy |

1. A method of preparing reconstituted 3D clusters comprising:
dissociating a tissue recovered from a subject into a mixture of dissociated individual cell types;
mixing the dissociated individual cell types in a liquid medium to form a mixture;
manipulating the mixture to aggregate the dissociated individual cell types into reconstituted 3D clusters, wherein each reconstituted 3D cluster comprises multiple cell types and the reconstituted 3D clusters comprises a microenvironment that mimics functionality, cell attachment and cell to cell interaction of the tissue.

2. The method of claim 1, wherein manipulating the mixture of dissociated individual cell types comprises applying an acoustic force

3. The method of claim 1, wherein manipulating the mixture of dissociated individual cell types comprises applying a centrifugation force.

4. The method of claim 1, wherein the tissue is a tumor tissue.

5. The method of claim 4, wherein the reconstituted 3D cell clusters comprise tumor cells and T-cells.

6. The method of claim 5, wherein the reconstituted 3D cell clusters comprise myeloid derived suppressor cells (MDSCs).

7. The method of any one of claim 1, wherein the reconstituted 3D cell clusters are embedded within a matrix.

8. The method of claim 7, wherein the liquid medium comprises a polymerizable component, and the method further comprises stabilizing the reconstituted 3D cell clusters by inducing the liquid medium to form the matrix after the dissociated individual cell types are aggregated into the reconstituted 3D cell clusters.

9. The method of claim 8, comprising inducing the polymerizable component to form a matrix by an alteration in temperature, pH, or by exposure to UV light.

10. The method of claim 1, further comprising a step of altering the mixture of dissociated individual cell types prior to the manipulating step, wherein the altering comprises:
i) removing one or more specific individual cell type from the mixture;
ii) changing the ratio of the cell types present in the mixture;
iii) adding a bioactive agent to the mixture;
iv) adding cells to the mixture; or v) any combination of two or more of i), ii), iii) and iv).

11. The method of claim 10, wherein the bioactive agent is an anti-tumor agent.

12. The method of any of claim 1, further comprising a step of treating the reconstituted 3D cell clusters with a second bioactive agent, exogenous cells, or an environmental manipulating agent.

13. An array of assembled three dimensional (3D) cell clusters, prepared from dissociated cells of a primary tissue of a subject, the 3D cell clusters comprising multiple cell types of the primary tissue, wherein the multiple cell types generate a microenvironment that mimics functionality, cell attachment, and cell to cell interaction of the primary tissue.

14. The array of claim 13, wherein the primary tissue is a tumor recovered from the subject, and wherein the 3D cell clusters comprise tumor cells, myeloid derived suppressor cells, and T-cells.

15. The array of claim 13, wherein acoustic waves are used to form the assembled 3D cell clusters.

16. The array of claim 13, wherein the 3D cell clusters are embedded within a matrix.

17. The array of claim 13, wherein the 3D cell clusters are about 150 μm to 250 μm.

18. An array of assembled three dimensional (3D) cell clusters of cells produced by the method of any one of claim 13.

19. A method of identifying cancer immunotherapy drugs comprising:
assembling an array of three dimensional (3D) cell clusters prepared from dissociated cells of a tissue of a subject, and
evaluating the function and response of one or more drugs in the array of three dimensional (3D) cell clusters,
wherein acoustic waves are used to form the array of 3D cell clusters.

20. The method of claim 19, wherein the tissue is a tumor tissue.
